(19) United States
(12) Patent Application Publication
(10) Pub. No.: US 2011/0098230 A1
(43) Pub. Date: Apr. 28, 2011

(54) COSMETIC USE OF ANNEXIN II-TYPE PROTEINS FOR TREATING DRYNESS OF THE SKIN

(75) Inventors: Dominique Bernard, Paris (FR); Lucie Simonetti, Vicennes (FR); Isabelle Castiel, Nice (FR)

(73) Assignee: L'Oreal, Paris (FR)

(21) Appl. No.: 12/936,320

(22) PCT Filed: Apr. 2, 2009
(86) PCT No.: PCT/FR09/50563

§ 371 Date: Jan. 3, 2011

Related U.S. Application Data
(60) Provisional application No. 61/049,004, filed on Apr. 30, 2008.

(30) Foreign Application Priority Data
Apr. 4, 2008 (FR) 08 52281

(51) Int. Cl. A61K 38/16; A61K 38/08; A61K 38/10; A61Q 19/00

(52) U.S. Cl. 514/18.8

(57) ABSTRACT

The present invention relates to the use, in particular cosmetic and/or therapeutic use, of annexin II, of polypeptides derived from this protein or of analogues thereof, of a nucleic sequence encoding such a polypeptide or of a modulator of the activity, the stability or the expression of such a polypeptide, in particular for preventing and/or treating the signs of skin dryness.

The invention also relates to the use of annexin II, of polypeptides derived from this protein or of analogues thereof, or of a nucleic sequence encoding such a polypeptide, as a marker for evaluating a state of dryness of an epithelium.

The present invention relates to the use, especially cosmetic and/or therapeutic use, of annexin II, of polypeptides derived from this protein, for example obtained from proteolysis of this protein, or of analogues thereof, of a nucleic sequence encoding such a polypeptide or of a modulator of the activity, the stability or the expression of such a polypeptide, for preventing and/or treating the signs of skin dryness and/or skin disorders associated with a state of skin dryness.

The invention also relates to the use of annexin II, of polypeptides derived from this protein or of analogues thereof, or of a nucleic sequence encoding such a polypeptide, as a marker for evaluating a state of dryness of an epithelium, and in particular of the epidermis.

The epidermis is an epithelium, conventionally divided into a basal layer of keratinocytes containing, in particular, skin stem cells and constituting the germinative layer of the epidermis, a “spiny” layer formed from several layers of polyhedral cells arranged on the basal layer, a “granular” layer comprising one to three layers of “flattened cells” containing distinct cytoplasmic inclusions, keratohyalin granules, and, finally, an assembly of upper layers, known as the horny layer (or stratum corneum) and formed from keratinocytes at the terminal stage of their differentiation, known as corneocytes.

As a result of its sturdiness and its compact stratified structure, the stratum corneum provides a barrier function: it in particular opposes transcutaneous water loss, also known as “transepidermal water loss”.

Thus, one of the functions of the stratum corneum is to take up and retain the water contained in the epidermis, and any impairment of its structure and/or its function may result in changes in the hydration of the skin.

The skin is hydrated by means of the water from the deep layers and by sweat.

An imbalance in skin hydration may result in profound consequences, both physiological and cosmetic.

Skin hydration disorders, and in particular skin dryness, are often observed with age. However, such states may also be manifested in young individuals.

The skin dryness state may be of nonpathological constitutional or acquired origin, or it may have a pathological constitutional origin.

Many external factors may lead to drying out of the skin or aggravate this state. Among these factors, mention may be made of climatic conditions such as cold or wind, sunlight, and exposure to certain chemical or therapeutic agents.

From a physiological point of view, dry skin is often associated with a lowering of the degree of hydration of the skin and also with a modification of the process of maturation of the stratum corneum, the most visible sign of which is the appearance of squamae at the surface of the skin.

From a sensory point of view, dry skin can be characterized by a sensation of skin tautness and/or tension.

Various methods exist for evaluating dry skin.

A first type of evaluation, performed from a strictly visual point of view, is based on a photographic atlas and uses a scale of 0 to 4 to note the score. The value 0 corresponds to an entirely normal skin, whereas the value 4 corresponds to a very dry skin. This method, which is subjective in nature, has the drawback of requiring the presence of visible cutaneous symptoms.

A second type of evaluation is based on biophysical measurements.

These methods are for the most part based on the electrical properties of the skin, i.e. the capacitance, the conductance and the impedance of the skin. This is the case, for example, for measuring the hydration index of the horny layer by corneometry, based on the ability of the skin to conduct an electric current. This can be carried out using various marketed instruments, such as the corneometer from the company Courage & Khazaka.

Finally, other types of evaluation, occasionally termed “morphometric” techniques, relate to the analysis of the microrelief of the skin or of the state of the cells at the skin surface, such as the evaluation of the “desquamation” by taking samples by “stripping” using adhesive of “D′squam” type from the company Cu-Derm.

These methods have the drawback of having to be combined together in order to give a reliable measurement of the state of dryness of the skin.

Moreover, they also have the drawback of not giving any indication regarding the origin of the dry skin and thus the means for correcting it.

Consequently, there is a need for a new marker, in particular a biological marker, that is capable of giving a reliable measurement of the state of an epithelium, and in particular a state of dryness.

There is therefore a need for new tools for promoting the integrity and maturation of the stratum corneum and also for evaluating its state.

There is also a need for new tools and/or new cosmetic and/or dermatological targets that can be used for the purposes of preventing and/or relieving the physiological and/or sensory signs associated with dryness of an epithelium, and in particular of an epidermis.

There is also a need for new cosmetic or dermatological targets for treating the state of dryness of an epithelium, and in particular of an epidermis.

There is also a need for new tools, in particular new molecules, for treating and/or preventing a state of dryness of an epithelium, and in particular of the epidermis.

The object of the present invention is to meet these needs.

The present invention results more particularly from the characterization, by the inventors, of an increase in the level of expression of the annexin II protein in the stratum corneum of a dry human epidermis, in comparison with the level in a stratum corneum of normal epidermis. In addition, more particularly, the invention comes from the observation of an increased expression of annexin II in aged and dry human epidermis, compared with a young epidermis or aged epidermis showing normal hydration.

Annexin II is a protein of 339 amino acids (SEQ ID No. 2) which, in order to perform its functions, can associate with the S100A10 protein so as to form a heterotetramer composed of two subunits of each of the proteins.

It belongs to a family of calcium-dependent phospholipid-binding proteins, indicating a plasma membrane localisation for annexins. Owing to this particular localisation, annexins have been allocated a role in the organisation of the events of membrane fusion, of exocytosis and endocytosis type. These proteins are involved in various signalling pathways, in particular in that of calcium.

Annexin II is a ubiquitous protein, the expression of which is particularly abundant in the skin. Owing to a calcium channel activity shown in vitro, it has been suggested that, during keratinocyte terminal differentiation, annexin II can play a part in regulating calcium flux.

In a study relating to skin ageing, Gromov et al. did not observe any variation in expression of the annexin II protein with age (Mol. Coll. Proteomics, 2003 February; 2(2): 70-84).

To the inventors' knowledge, annexin II had never been identified hitherto as being a protein of which the expression varies according to the skin typology, i.e. an increased level of expression in a dry human stratum corneum compared with that present in a normal human stratum corneum.

Thus, contrary to all expectation, annexin II also proves to be a potential marker for the physiological state of the skin, in particular in terms of dryness. Specifically, as emerges from the tests featured hereinafter, the inventors have noted, unexpectedly, firstly an expression of this protein in the stratum corneum, and secondly a significant increase in its expression in a stratum corneum sampled from a dry skin, in comparison with a stratum corneum sampled from a normal skin.

Consequently, according to one of its first aspects, a subject of the present invention is a cosmetic or alternatively nontherapeutic use of an effective amount of at least one polypeptide derived from annexin II, and in particular having an amino acid sequence encoded by a nucleic acid sequence wholly or partially represented by a sequence represented by SEQ ID No. 1, an analogue thereof or a fragment thereof, of at least one nucleic sequence encoding such a polypeptide or of at least one modulator of the activity, the stability or the expression of such a polypeptide, as an agent that is useful for preventing and/or treating the signs of skin dryness, in particular for preventing and/or treating dehydration of an epidermis.

According to another of its aspects, a subject of the present invention is also the use of an effective amount of at least one polypeptide derived from annexin II, and in particular having an amino acid sequence encoded by a nucleic acid sequence wholly or partially represented by a sequence represented by SEQ ID No. 1, an analogue thereof or a fragment thereof, of at least one nucleic sequence encoding such a polypeptide or of at least one modulator of the activity, the stability or the expression of such a polypeptide, for the preparation of a composition, in particular a therapeutic composition, intended for preventing and/or treating the signs of skin dryness, in particular for preventing and/or treating dehydration of an epidermis.

The term “signs of skin dryness” is intended to mean not only all the modifications of the outer appearance of the skin due in particular to the dehydration of the epidermis, such as the rough and flaky appearance, and also the decreased suppleness, but also the sensations associated with the phenomenon of dryness, such as itching and/or tautness. It may, in fact, occur that these sensations are felt by an individual without any visual symptom necessarily being perceptible.

According to one embodiment of the invention, a use in accordance with the invention may be more particularly intended for preventing and/or treating the signs of nonpathological constitutional or acquired skin dryness.

For the purpose of the present invention, the expression “effective amount” is intended to denote the minimum amount required for observation of the expected effect, i.e. a cosmetic effect or a therapeutic effect, it being understood that the effective amounts required for obtaining a cosmetic effect or a therapeutic effect may be, as appropriate, identical or different.

For the purpose of the invention, the term “cosmetic use” is intended to denote a use intended mainly for providing an aesthetic effect and/or comfort.

For the purpose of the invention, the term “therapeutic composition” is intended to denote a composition intended for providing a prophylactic or curative effect with regard to epithelial and in particular epidermal disorders, recognized as reflecting a pathological state.

For the purpose of the invention, the term “prophylactic” or “preventive” is intended to mean a reduction of the risk of occurrence of a phenomenon, for example a pathological condition.

A composition in accordance with the invention may in particular be intended for preventing and/or treating dryness of an epidermis, and in particular a defect of hydration of the stratum corneum.

A composition in accordance with the invention may in particular be intended for preventing and/or treating itching and/or tautness sensations in a dry epithelium.

According to yet another of its aspects, the present invention also relates to the use of at least one polypeptide in accordance with the invention, or at least one nucleic acid sequence encoding said polypeptide, as a tool for the in vitro or ex vivo characterization of a state of dryness of an epithelium, and in particular of an epidermis.

More specifically, according to another of its aspects, the present invention relates to a process, in particular a cosmetic, noninvasive process, for characterizing the state of dryness of the surface of an epithelium, in particular of an epidermis, comprising at least the qualitative or quantitative characterization of the expression and/or of the biological activity of a polypeptide in accordance with the invention, i.e. annexin II, or of a derivative or fragment thereof.

According to one embodiment variant, the datum or value obtained may be assessed in comparison with a reference datum or value, obtained, for example, from at least one epithelium, in particular an epidermis, different from that which is the subject of the characterization, and the state of which is known. By way of example of a reference epidermis, it may be either an epidermis of a second individual who has normal skin distinct from a first individual on whom the characterization is performed, or a region of the epidermis of the same individual on whom the characterization is performed, but chosen from an area of the skin that shows physiological hydration.

According to another of its aspects, the present invention is also directed towards a process, in particular a cosmetic, noninvasive process, for characterizing the effectiveness of a cosmetic or therapeutic treatment aimed at compensating for the signs of skin dryness, comprising at least the qualitative or quantitative characterization of the expression and/or of the biological activity of a polypeptide in accordance with the invention, i.e. annexin II, or of a derivative or fragment thereof.

According to one embodiment variant, the datum obtained at the end of the characterization may also be examined in comparison with a reference value or datum. This reference value or datum may be a datum obtained from the epithelium, in particular from the epidermis, to be subjected to the treatment, prior to the administration of said treatment or in a shorter chronological delay with regard to the treatment start date.

As emerges from the description which follows, the processes according to the invention are particularly advantageous insofar as their implementation does not require an invasive procedure.

The processes of the invention can be carried out in vitro, ex vivo or in vivo.

Specifically, the localisation, by the inventors, of the novel dryness biomarker, namely annexin II, in the stratum corneum permits a quantitative or qualitative characterization of the expression of this protein by simple topical sampling. The sampling method may be, for example, a technique of “stripping” type, consisting in applying to the epithelium under consideration, such as an epidermis, a portion of adhesive tape. On detaching this adhesive tape, a fraction of the epithelium, for example an epidermal fraction, is removed. After protein extraction, said fraction is analysed by conventional methods, such as immunoenzymatic assay or, more particularly, Western blot analysis.

Dry Skin

A dry skin has a rough feel, appears covered with squamae and manifests itself essentially through a sensation of tautness and/or tension.

Dry skin is in fact accompanied by a desquamation disorder and presents various stages as a function of the severity of this desquamation. When the skin is slightly dry, these squamae are abundant but sparingly visible to the naked eye, and removal takes place corneocyte by corneocyte. They are increasingly infrequent but increasingly visible to the naked eye when this disorder worsens, and the patches may comprise several hundred corneocytes, thus representing more or less large patches, known as squamae.

The origin of this dryness may be of constitutional or acquired type.

In the case of constitutional dry skin, two categories can be distinguished: pathological skin and nonpathological skin.

Pathological constitutional dry skin is essentially represented by atopic dermatitis and ichthyosis. It is virtually independent of the external conditions, and arises from known or unknown genetic modifications. Among the known genetic modifications that affect skin hydration, mention may be made, for example, of modifications of the transglutaminase 1 gene or those of the filaggrin gene.

In the case of nonpathological constitutional dry skin, the severity of the state of dryness may, for its part, depend on external factors. Senile skin (characterized by a general decrease in metabolism in the skin with age), fragile skin (very sensitive to external factors and often accompanied by erythema and rosacea) and xerosis vulgaris (of probable genetic origin and mainly manifesting itself on the face, the limbs and the back of the hands) are included in this skin category.

In the case of acquired dry skin, the involvement of external parameters such as exposure to chemical agents, to harsh climatic conditions, to sunlight or else to certain therapeutic treatments (for example retinoids) is a determining factor. Under these external influences, the epidermis may then become momentarily and locally dry. This may concern any type of epidermis.

Irrespective of the origin, a skin suffering from skin dryness generally displays the following signs, namely a rough and flaky feel, and also decreased suppleness and elasticity.

Dry skin, also known as “xerosis”, can appear at any age and is unconnected to a pathological condition. In this case, it will be referred to as “acquired” dryness.

However, xerosis becomes more frequent and debilitating with age, in particular in women. This is then referred to as senile xerosis. Moreover, women generally suffer a worsening of skin dryness during the menopause, probably due to the hormonal dysregulation characteristic of this phenomenon. The areas most affected are the lower legs, the back of the forearms and the hands.

As previously mentioned, acquired dryness may be subject to the influence of external factors. For example, the appearance of dry skin may be promoted by cold, dry, wintery weather. This is then referred to as winter xerosis. Skin dryness may also be induced by an exogenous stress, of chemical origin, for example of anionic detergent type, or alternatively of mechanical origin (chafing or shaving).

Although no study has demonstrated an incidence of dryness on the origin and formation of wrinkles and fine lines, which are essentially attributable to ageing, in visual terms a dry skin makes said wrinkles and fine lines more apparent.

Moreover, from a sensory viewpoint, skin dryness is characterized by a sensation of tautness and/or itching. For obvious reasons, these manifestations are not only sources of discomfort, or even pain, but also an unaesthetic appearance.

Thus, there remains a need for novel active agents capable of exerting a beneficial action in the treatment of skin dryness, not only from a therapeutic viewpoint, but also from an aesthetic viewpoint.

Definition of Polypeptide

According to one embodiment, a polypeptide suitable for the invention can have an amino acid sequence wholly or partially represented by a sequence represented by SEQ ID No. 2, an analogue thereof or a fragment thereof.

For the purpose of the present invention, the term “annexin II” is intended to denote, in general, unless otherwise indicated, the sequence (SEQ ID No. 2) of the protein optionally having undergone post-translational modifications.

Among these post-translational modifications, mention may in particular be made of phosphorylations on the amino acids in positions 18, 19, 24, 26 and 30 of the protein.

It is, moreover, known that the primary sequence of a polypeptide, i.e. the sequence of the amino acids, determines sites specifically recognised by enzymes of protease type, such as trypsin, which, once these sites have been recognised, will induce the cleavage of the polypeptide by proteolysis. This proteolysis results in the generation of various peptides, or proteolytic fragments, of annexin II.

The inventors have detected the presence of such peptides in the stratum corneum.

Consequently, the invention also extends to the proteolytic fragments of annexin II.

Thus, according to one particular embodiment, a polypeptide suitable for the invention can have an amino acid sequence chosen from SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42 and SEQ ID No. 43, and mixtures thereof.

The term “analogue of a polypeptide” is intended to denote any polypeptide which exhibits a sequence homology, in particular with respect to one of the characteristic sequences of said polypeptide, and also a biological activity of the same nature.

This compound may be a peptidomimetic agent.

The homology may be at least 85%, for example at least 90%, and for example at least 95%. The homology can be determined by visual comparison or by means of any computer tool generally used in the field, such as the BLAST programs available at www.ncbi.nlm.nih.gov and used with the default parameters.

The sequence homology can result from modifications derived from mutation or variation in the sequences of the peptides according to the invention, originating either from the deletion or insertion of one or more amino acids, or from the substitution of one or more amino acids in the characteristic sequences of a polypeptide according to the invention.

For the purpose of the invention, the term “polypeptide fragment” is intended to denote any portion of a polypeptide in accordance with the invention comprising at least 4, at least 6, in particular at least 8, and more particularly at least 12 consecutive amino acids of said polypeptide, and a substantially similar biological activity.

The term “characteristic sequence of the polypeptide” is intended to denote, in particular with regard to annexin II, the sequence represented by SEQ ID No. 2.

In general, the polypeptide analogues can comprise conservative substitutions relative to the amino acid sequence of the natural polypeptide.

Several of these modifications may be combined.

By way of example of mutations that can be considered in the present invention, mention may be made, in a nonexhaustive manner, of the replacement of one or more amino acid residues with amino acid residues having a similar hydropathic index, without, however, substantially affecting the biological properties of the polypeptide, and in particular its biological activity, such as its activity of stimulating the proliferation and/or migration and/or terminal differentiation of keratinocytes or of stimulating the synthesis of proteoglycans in an epithelium, and in particular the epidermis.

The hydropathic index is an index attributed to amino acids as a function of their hydrophobicity and their charge (Kyte et al. (1982), J. Mol. Biol., 157:105).

A polypeptide or analogue also covered by the present invention may be a polypeptide that has undergone one or more post-translational modification(s).

The term “post-translational modification(s)” is intended to encompass all the modifications that a peptide or a protein is capable of undergoing after its synthesis in a cell, for instance one or more phosphorylation(s), one or more thiolation(s), one or more acetylation(s), one or more glycosylation(s), one or more lipidation(s), such as a farnesylation or a palmitoylation, a structural rearrangement such as disulphide bridge formation and/or such as cleavage within the peptide sequence.

The analogue moreover has substantially the same biological activity as the natural polypeptide.

According to one embodiment, a polypeptide suitable for the implementation of the invention may also be a natural or synthetic polypeptide, which, as appropriate, can be obtained after enzyme or chemical lysis of annexin II or by chemical or biological synthesis or by extraction from a biological tissue, for instance the skin, naturally expressing this polypeptide or expressing it after transfection thereof, and also the various post-translational forms thereof, or else any natural or synthetic polypeptide of which the sequence wholly or partially comprises (in whole or in part) an abovementioned amino acid sequence, for example the variants and the analogues.

Those skilled in the art can obtain a polypeptide in accordance with the invention by means of recombinant DNA-based processes, for instance those described in the manual “Molecular Cloning—A Laboratory Manual” (2^{nd} edition), Sambrook et al., 1989, Vol. I-III, Cold Spring Harbor Laboratory, Cold Spring Harbor Press, N.Y. (Sambrook).

According to another embodiment, a polypeptide suitable for the implementation of the invention may also be a polypeptide as defined above, in which at least one residue has been replaced with an amino acid residue having a similar hydropathic index, as defined above.

According to another embodiment, a polypeptide suitable for the implementation of the invention may also be a polypeptide as defined above, fused with another polypeptide, a hydrophilic or hydrophobic targeting agent, a bioconversion precursor, or a luminescent, radioactive or colorimetric label.

In a nonlimiting manner, as examples of compounds that may be coupled with a polypeptide in accordance with the invention, mention may be made of fluorescent proteins such as the Green Fluorescent Protein, fluorescent chemical compounds such as rhodamine, fluorescein or Texas Red®, phosphorescent compounds, radioactive elements, such as ³H, ¹⁴C, ³⁵S, ¹²¹I or ¹²⁵I, or colorimetric labels, for instance chromogenic substrates sensitive to the action of galactosidase, peroxidase, chloramphenicol acetyl transferase, luciferase or alkaline phosphatase.

Depending on the nature of the compounds that may be coupled with a polypeptide in accordance with the invention, the coupling can be carried out via chemical processes, in particular by means of reactive chemical functions, or via molecular biology processes known to those skilled in the art.

Definition of Nucleic Acid Sequences

According to one embodiment, the present invention also relates to nucleic acid sequences encoding a polypeptide of the invention and to the employment thereof in the various uses and processes in accordance with the invention.

Thus, the present invention also relates to the use of nucleic acid sequences, in particular deoxyribonucleic acid or ribonucleic acid sequences, encoding a polypeptide in accordance with the invention, in particular the sequences corresponding to at least one nucleic acid sequence represented by SEQ ID No. 1, analogues thereof or a fragment thereof, for the preparation of a composition in accordance with the invention.

For the purpose of the present invention, the term “nucleic acid sequence fragment” is intended to denote a nucleic acid sequence encoding all or part of a polypeptide in accordance with the invention, or an analogue thereof, and in particular a nucleic acid sequence represented by SEQ ID No. 1 or an analogue thereof.

The term “analogue of a nucleic acid sequence” is intended to denote any nucleic acid sequence, optionally resulting from the degeneracy of the nucleic acid code, and encoding a polypeptide having a sequence identical or analogous to that of the polypeptide encoded by said nucleic acid sequence.

The nucleic acid sequences may be derived from any possible origin, i.e. either animal, in particular from mammals and even more particularly humans, or plant origin, or from microorganisms (viruses, phages, bacteria, inter alia) or else from fungi, without prejudice as to whether or not they are naturally present in said organism of origin.

In the present case, the invention also relates to the use of isolated and purified nucleic acid fragments encoding the polypeptides under consideration according to the invention.

A nucleic acid sequence in accordance with the invention may comprise a sense, antisense or interfering sequence corresponding to a sequence encoding a polypeptide in accordance with the invention.

Thus, the present invention also relates to the use of nucleic acid sequences, in particular deoxyribonucleic or ribonucleic acid sequences, encoding a polypeptide in accordance with the invention.

The nucleic acid sequences according to the invention may in particular be used for preparing the corresponding sense or antisense ribonucleic acid sequences.

A subject of the invention is also the use of any polynucleotide, having a ribonucleic acid or deoxyribonucleic acid sequence, comprising a sense or antisense sequence, in particular small interfering RNA (siRNA), corresponding at least to the nucleic acid sequence SEQ ID No. 1 or an analogue thereof.

Modulator

According to another embodiment, the invention relates to the use of a modulator of the expression and/or the stability and/or the activity of a polypeptide in accordance with the invention.

For the purpose of the invention, the term “modulator” is intended to mean, in relation to a given effect, the action of stimulating or inhibiting this effect.

For the purpose of the present invention, the expression “modulator or chemical or biological compound capable of modulating the biological activity and/or the expression” is intended to mean any compound capable of acting, directly or indirectly, on at least one polypeptide in accordance with the invention, or a nucleic acid sequence encoding the latter, or on an element of an intracellular or extracellular signalling pathway, or of a metabolic pathway, involving said polypeptide, or on an element involved in regulating the transcription and/or the translation of a nucleic acid sequence encoding said polypeptide, and also in regulating the stability thereof.

The term “biological activity” is intended to denote, in particular in relation to annexin II, the biological activity of the protein represented by the sequence SEQ ID No. 2.

This modulator may be an agent for activating or inhibiting the expression of a polypeptide of the invention, or else an agent for regulating the stability of said polypeptide.

By way of nonlimiting illustration, among the agents for regulating the stability, mention may in particular be made of compounds for stimulating proteolytic degradation, such as proteases, ion chelators, sulphonic derivatives, urea derivatives, reducing agents, alpha- or beta-hydroxy acids, ascorbic acid or nicotinamide.

More particularly, the modulator may be an inhibitor of the expression of the polypeptides according to the invention.

According to one preferred embodiment, the modulator is an agent for reducing the stability of the polypeptides in accordance with the invention, by stimulating the proteolytic degradation thereof.

It is understood that all of the cosmetic or therapeutic compositions under consideration according to the invention employ a physiologically acceptable medium.

For the purposes of the present invention, the term “physiologically acceptable medium” is intended to denote a medium which is suitable for the application of a composition to an epithelium or a keratin material, such as the skin, the scalp, the lips, the mucous membranes and keratin fibres such as the hair, the nails and body hairs, or, where appropriate, orally or parenterally.

For the purpose of the present invention, the term “therapeutic” is intended to denote a composition which can be used in the context of a prophylactic and/or curative treatment, or of a method for evaluating a state of dryness of an epithelium, and in particular of the epidermis.

According to another embodiment, a cosmetic or therapeutic composition in accordance with the invention can also comprise at least one cosmetic and/or therapeutic active agent.

As examples of active agents that can be used in the context of the present invention, mention may be made of cosmetic oils, such as silicone oils, plant oils of triglyceride type, hydrocarbon-based oils such as parleam oil, and esters of fatty acids and of fatty alcohols.

It may also be possible to use other active agents which make it possible to improve the condition of the skin, such as hydrating or moisturising active agents or active agents which make it possible to improve the natural lipid barrier, such as ceramides, cholesterol sulphates and/or fatty acids, and mixtures thereof.

It may also be possible to use enzymes which have an activity on the skin, such as proteases, lipases, glucosidases, amidases, cerebrosidases and/or melanases, and mixtures thereof.

As other examples of active agents suitable for implementing the present invention, mention may be made of: analgesic active agents, antiyeast active agents, antibacterial active agents, antiparasitic active agents, antifungal active agents, antiviral active agents, steroidal anti-inflammatory active agents, anaesthetic active agents, antipruritic active agents, keratolytic active agents, free-radical scavenger active agents, antiseborrheic active agents, antidandruff active agents, anti-acne active agents, active agents intended for preventing ageing of the skin and/or for improving the condition thereof, anti-dermatitis active agents, antiirritant active agents, immunomodulatory active agents, active agents for the treatment of dry skin, antiperspirant active agents, antipsoriatic active agents, active agents for protecting against UV, antihistamine active agents, cicatrising active agents, self-tanning active agents, antioxidants such as green tea or active fractions thereof, glycerol, laponite, caffeine, aromatic essential oils, colorants, depigmenting active agents, liporegulators, emollient, refreshing, deodorising, desensitising, bleaching or nourishing active agents, active agents for reducing skin differentiation and/or proliferation and/or pigmentation, and mixtures thereof.

In general, any composition of the invention may be applied to the skin (on any skin region of the body) or to the mucous membranes (buccal, jugal, gingival, genital, conjunctival, etc.).

In a known manner, a cosmetic composition may also contain adjuvants which are customary in the cosmetics field, such as hydrophilic or lipophilic gelling agents, hydrophilic or lipophilic additives, preservatives, antioxidants, solvents, fragrances, fillers, screens, odour absorbers and dyestuffs.

The amounts of the various constituents of the compositions according to the invention are those conventionally used in the fields under consideration.

The amount of chemical or biological compound or of polypeptide, nucleic acid sequence or modulator in accordance with the invention contained in a composition according to the invention, also referred to as “effective amount”, of course depends on the nature of the compound and on the desired effect, and can therefore vary to a large extent.

To give an order of magnitude, a composition may contain a modulator in accordance with the invention or a polypeptide in an amount representing from 0.00001% to 50% of the total weight of the composition, in particular in an amount representing from 0.001% to 10% of the total weight of the composition, and more particularly in an amount representing from 0.1% to 1% of the total weight of the composition.

A composition according to the invention may be more particularly intended for reducing and/or treating a hydration deficiency, and in particular a dryness, that may cause deterioration of the state of an epithelium, and in particular of an epidermis.

According to another aspect, the present invention also relates to the use of at least one polypeptide in accordance with the invention or of at least one nucleic acid sequence encoding said polypeptide, as a tool for characterizing, in vitro or ex vivo, a state of dryness of an epithelium, and in particular of an epidermis.

Thus, according to another of its aspects, the present invention relates to noninvasive processes for characterizing the state of dryness of the surface of a nonpathological epidermis or else the effectiveness of a cosmetic or therapeutic treatment directed at qualitatively or quantitatively characterizing the expression of annexin II, or of a derivative or fragment thereof.

These processes are particularly advantageous since their implementation does not require obligatory recourse to a surgical technique for carrying out such a characterization. An extract of the epidermis can thus be obtained by simple stripping and directly analysed by a conventional analytical technique, in particular as described above.

According to one embodiment, a process for characterizing a state of dryness of an epithelium, for example an epidermis, comprises at least the steps consisting in:
a) determining, in a sample of said epithelium, the content of a polypeptide in accordance with the invention, or of a nucleic acid sequence encoding said polypeptide, and
b) comparing said content determined in step a) to a reference value.

Advantageously, a process of the invention is noninvasive.

A process of the invention is advantageously carried out on an isolated sample.

According to one embodiment, a process according to the invention can be carried out on a sample of epithelium, and in particular of epidermis, taken from an individual.

A process according to the invention may also be carried out on a sample of epithelium, and in particular of epidermis, taken from an epithelial cell model, and in particular an epidermal cell model, or from a reconstructed isolated skin, in order to define the state thereof.

A sample of epithelium may be taken by any method known to those skilled in the art.

A process according to the invention may be carried out in vivo, in vitro or ex vivo.

A reference value may, for example, be a content of polypeptide or of nucleic acid sequence determined on a sample of epidermis taken from an epithelium, and in particular from normal skin, i.e. skin that is satisfactory from a physiological point of view, like, for example, a hydrated skin.

A reference value may be measured in parallel with or following the determination of said content of a polypeptide or of a nucleic acid sequence.

A comparison of a determined content with a reference value may make it possible to evaluate a deviation relative to this value.

The analysis of the intensity and/or of the nature of this deviation (negative or positive) may be informative with regard to the state of the epidermis.

The characterization of a state of dryness of an epidermis may be indicative of a possible skin disorder which can be corrected through the use of compounds capable of modulating the expression of a polypeptide of the invention.

According to one embodiment, a process according to the invention may be implemented in a process for the in vivo, in vitro or ex vivo diagnosis of dryness of an epithelium, and in particular of the epidermis, in an individual.

A polypeptide suitable for carrying out a process according to the invention can advantageously be annexin II.

The determination of the content of polypeptide in accordance with the invention or of nucleic acids in accordance with the invention in a sample of epidermis may be carried out by means of any protocol known to those skilled in the art.

The expression of a nucleic acid sequence can be determined, for example, by means of oligonucleotide probes, using any protocol known to those skilled in the art.

By way of examples of methods for detecting a nucleic acid sequence, mention may be made of the quantitative (Q-PCR) or nonquantitative polymerase chain reaction (PCR), in the presence or absence of reverse transcriptase (RT-PCR or Q-RT-PCR), of Northern blotting, of the ribonuclease protection assay method, of methods with DNA chips, of methods with transcriptome chips, of methods with oligonucleotide chips, and of in situ hybridization methods.

By way of example of agents suitable for the detection of a nucleic acid sequence, and in particular of mRNA, mention may be made of labelled nucleic acid probes that can hybridise to said sequence.

Such a nucleic acid probe can be readily obtained by any method known to those skilled in the art.

Thus, the nucleic acid sequences in accordance with the invention may be used to prepare sense and/or antisense oligonucleotide primers, which hybridise, under high stringency conditions, to the sequence SEQ ID No. 1 or an analogue thereof.

The expression of a nucleic acid sequence in accordance with the invention can be compared to a reference value obtained, for example, by carrying out a process in accordance with the invention in the absence of test compound.

The expression of a nucleic acid sequence can also be determined, indirectly, by determining the expression of the polypeptide encoded by said sequence, by means of any technique known in the field, such as Western blotting, ELISA, the Bradford or Lowry method, or as indicated hereinafter.

A nucleic acid sequence suitable for carrying out a process according to the invention may advantageously be a nucleic acid sequence encoding annexin II, for example of mRNA type.

The determination of the content of a polypeptide in accordance with the invention can be carried out by means of any method known to those skilled in the art.

By way of examples of methods for detecting a polypeptide, mention may be made of Western blotting, slot blotting, dot blotting, ELISA (Enzyme Linked ImmunoSorbent Assay) methods of the singleplex or multiplex type, proteomics or glycomics methods, staining of polypeptides in a polyacrylamide gel with a silver-based stain, with Coomassie blue or with SYPRO, immunofluorescence, UV absorption, immunohistochemical methods in conventional, electron or confocal microscopy, FRET (fluorescence resonance energy transfer), TR-FRET (time-resolved FRET) methods, FLIM (fluorescence lifetime imaging microscopy) methods, FSPIM (fluorescence spectral imaging microscopy) methods, FRAP (fluorescence recovery after photobleaching) methods, reporter gene methods, AFM (atomic force microscopy) methods, surface plasmon resonance methods, microcalorimetry methods, flow cytometry methods, biosensor methods, radioimmunoassay (RIA) methods, isoelectric focusing methods, and enzyme assays, methods using peptide chips, sugar chips, antibody chips, mass spectrometry methods, and SELDI-TOF spectrometry methods (Ciphergen).

The processes in accordance with the invention can be carried out on a sample, for example an isolated sample, of epithelium, in particular of epidermis, obtained from a skin biopsy or from an epithelial cell model, for example an epidermal cell model, or more advantageously from a noninvasive surface removal, in particular with adhesive (tape stripping), of stratum corneum or by simple washing.

A sample of epidermis can be taken by any method known to those skilled in the art.

These methods may be carried out by “stripping” techniques.

These strippings are sticky surfaces applied to the surface of the epidermis, such as Blenderm® from 3M, D'squam (commercial adhesive from CuDERM), cyanoacrylate glue or the varnish stripping method. By virtue of these strippings, the adherent corneocytes and the content of their intercellular spaces can be sampled and subsequently subjected to an extraction which makes it possible to access the protein content.

The taking of a sample suitable for the process can also be carried out more directly by “washing” the skin surface by means, for example, of accessories of the vane turbine type, of the spiral cell type (as described in patent FR 2 667 778) combined with a fluid circuit, or simply by addition/removal of a drop of buffer at the surface of the skin.

By way of indication, other sampling methods suitable for implementing the invention may be mentioned, such as methods based on scraping the upper part of the stratum corneum by means of a twin blade system. This technique makes it possible to collect squamae which can then be directly analysed by various techniques in order to determine the mineral, amino acid or lipid contents.

It is equally possible to envisage detecting the presence of a polypeptide in accordance with the invention by means of an antibody, where appropriate in a labelled form.

An antibody that can be used as a tool for evaluating a state of an epidermis can be obtained by any process known to those skilled in the art, as described in “Antibodies: A Laboratory Manual”, Cold Spring Harbor Laboratory Press, Cold Spring Harbor, N.Y. (1990). Advantageously, the antibodies used may be recombinant antibodies, such as those developed by the company Antibodies-by-design.

The present invention also relates to a nontherapeutic process for demonstrating an effect of a treatment capable of causing regression of signs of dryness of an epithelium, in particular itching and/or tautness sensations, in an individual, comprising at least the steps consisting in:
a) carrying out, before the treatment, at least a first determination, in a first sample of an epithelium taken from said individual, of a biological activity and/or of the expression of a polypeptide in accordance with the invention, or of the expression of a nucleic acid sequence encoding said polypeptide,
b) carrying out, after the treatment, at least a second determination, in a second sample of an epithelium taken from said individual, of said biological activity and/or of said expression of said polypeptide or of said expression of said nucleic acid sequence, determined in step a), and
c) comparing the first and second determinations, in particular in order to deduce therefrom information relating to at least one effect of the treatment.

Such a treatment may in particular be a cosmetic treatment.

In particular, the treatment of which the effect is to be evaluated may be a treatment intended for relieving or reducing the signs of skin dryness.

The biological activity of a polypeptide known to those skilled in the art, for example and in a nonlimiting manner, mention may be made of methods of cell culture followed by characterization of differentiation markers, such as, for example, keratin 10 or filaggrin, or of proliferation markers, such as, for example, KI 67 and PCNA.

Advantageously, a polypeptide used in a process according to the present invention may be annexin II.

The expression of a polypeptide can be determined as indicated above.

According to another aspect, the present invention relates to a process for cosmetic treatment of the signs of skin dryness, comprising at least one step consisting in applying, to at least one part of the skin, the mucous membranes and/or the keratin fibres, at least one cosmetic composition in accordance with the invention.

For the purpose of the present invention, “a” should be understood, unless otherwise indicated, in the sense of “at least one”.

The examples presented hereinafter are given by way of nonlimiting illustration of the invention.

### EXAMPLE I

Analysis of the Expression of Annexin II in a Dry Stratum Corneum Versus a Normally Hydrated Stratum Corneum

The analyses are carried out using samples taken by varnish strippings performed on the legs of various individuals.

The individuals participating in the study are put into 4 groups.

The DA group corresponds to group 1: dry menopausal, n=15.

The NA group corresponds to group 2: normal menopausal, n=13.

The DY group corresponds to group 3: dry young, n=16. The NY group corresponds to group 4: normal young, n=14.

The selection of the individuals in one or other group is done on the basis of the visual evaluation, by an expert, of the state of skin dryness of each individual on the legs.

The comparison with a photographic atlas made it possible to assign to each individual a clinical score for skin dryness according to the following magnitude and scale:
score 0: normal skin; the skin exhibits a regular cutaneous relief and a smoothe appearance,
score 1: dehydrated skin; the skin exhibits a striated cutaneous relief and quite a rough appearance,
score 3: very dry skin; the skin exhibits numerous squamae and some scales, and also a coarse appearance, and
score 4: extremely dry skin; the skin exhibits a very large number of scales and a very coarse appearance.

1: Preparation of Acetone Powders

Two varnish strippings (B. Mehul et al., J. Biol. Chem. 2000, Apr. 28; 275(17): 12841-7) of 10 cm² are placed in 20 ml of acetone. The corneocytes become detached. The mixture is filtered through a 40 μm nylon membrane. Three successive rinses are carried out with the same volume of acetone. The suspension is finally filtered on a vacuum pump. Acetone powders of stratum corneum are obtained in the dry form.

2: Sample Extraction

An extraction is carried out under denaturing conditions. To do this, a prewash is carried out with a volume (100 μl) of PBS (phosphate buffered saline) buffer +0.1% Triton X100, which is added per mg of acetone powder. The mixture is ground in a Potter and centrifuged. The corneocyte pellet is collected. It is extracted with the same volume (100 μl/mg) of Laemmli buffer containing 0.0625 mM Tris, pH 6.8, 200 mM DTT, 2% SDS and 10% glycerol. The mixture is heated at boiling temperature for 10 minutes and is then ground and centrifuged for 10 minutes at 10 000 g. The supernatant is collected. A protein assay is carried out according to the Bradford technique with the Bradford reagent (Bio-Rad Protein assay). The samples are adjusted to 1 mg/ml.

3: Protein Analysis by Western Blotting

The proteins are separated by SDS-PAGE electrophoresis. After semi-dry blotting onto a PVDF membrane (Immobilon-P Millipore) according to a standard protocol, the proteins are incubated with the anti-annexin II primary antibody (BD Biosciences, 610068) overnight at 4° C. The second incubation is then carried out with the secondary antibody (goat anti-mouse IgG-HRP conjugate; Bio-Rad) directed against the primary antibody, for 1 h 30 at ambient temperature. The presence of annexin II on the membrane is revealed by immunodetection using the ECL plus kit (Amersham). The membrane is then stained with amido black in order to detect the total proteins present on the membrane. The image is acquired with FluorSmax (Biorad) and the bands are quantified using the Quantity-one software (Biorad).

4: Results

The results are expressed as delta cnt*mm² of the protein of interest/delta cnt*mm² of total proteins.

Methodology:
2-Way (age and type of skin) analysis of variance taking into account the interaction of these two factors+1-way (group) analysis of variance and Tukey's multiple comparison test. Since the normality and homoscedasticity conditions were not verified, the analysis was carried out after logarithmic transformation.

The statistical analysis was carried out with the SAS version 8.2 and SPSS version 12 software packages.

All the tests were carried out at the 5% two-sided threshold.

The table below gives the mean results and also their standard errors of the mean (sem).

| | | sem |
|---|---|---|
| Group | Annexin II | Annexin II |
| DA | 441 | 165 |
| NA | 154 | 21 |
| DY | 221 | 55 |
| NY | 135 | 29 |

A significant variation in the expression of annexin II according to the skin typology is noted: specifically, its expression is significantly increased in the “dry skin” groups (DY and DA), compared with the “normal skin” groups (NY and NA) (p=0.006).

Sequence Listing

SEQ ID NO 1

| | |
|---|---|
| 1 | gggacgctct cagctctcgg cgcacggccc aggttatctt gtagcatagc aacttcggat |
| 61 | ttcactctac ccggagagtt tcccgcttgg ttgaacacat tggcctcagg aagcttcctt |
| 121 | caaaatgtct actgttcacg aaatcctgtg caagctcagc ttggagggtg atcactctac |
| 181 | acccccaagt gcatatgggt ctgtcaaagc ctatactaac tttgatgctg agcgggatgc |
| 241 | tttgaacatt gaaacagcca tcaagaccaa aggtgtggat gaggtcacca ttgtcaacat |
| 301 | tttgaccaac cgcagcaatg cacagagaca ggatattgcc ttcgcctacc agagaaggac |
| 361 | caaaaaggaa cttgcatcag cactgaagtc agccttatct ggccacctgg agacggtgat |
| 421 | tttgggccta ttgaagacac ctgctcagta tgacgcttct gagctaaaag cttccatgaa |
| 481 | ggggctggga accgacgagg actctctcat tgagatcatc tgctccagaa ccaaccagga |
| 541 | gctgcaggaa attaacagag tctacaagga aatgtacaag actgatctgg agaaggacat |
| 601 | tatttcggac acatctggtg acttccgcaa gctgatggtt gccctggcaa agggtagaag |
| 661 | agcagaggat ggctccgtca ttgattatga actgattgac caagatgctc gggatctcta |
| 721 | tgacgctgga gtgaagagga aaggaactga tgttcccaag tggatcagca tcatgaccga |
| 781 | gcggagcgtg ccccacctcc agaaagtatt tgataggtac aagagttaca gcccttatga |
| 841 | catgttggaa agcatcagga aagaggttaa aggagacctg gaaaatgctt tcctgaacct |
| 901 | ggttcagtgc attcagaaca agcccctgta ttttgctgat cggctgtatg actccatgaa |
| 961 | gggcaagggg acgcgagata aggtcctgat cagaatcatg gtctcccgca gtgaagtgga |
| 1021 | catgttgaaa attaggtctg aattcaagag aaagtacggc aagtccctgt actattatat |
| 1081 | ccagcaagac actaagggcg actaccagaa agcgctgctg tacctgtgtg gtggagatga |
| 1141 | ctgaagcccg acacggcctg agcgtccaga aatggtgctc accatgcttc cagctaacag |
| 1201 | gtctagaaaa ccagcttgcg aataacagtc cccgtggcca tccctgtgag ggtgacgtta |
| 1261 | gcattacccc caacctcatt ttagttgcct aagcattgcc tggccttcct gtctagtctc |
| 1321 | tcctgtaagc caaagaaatg aacattccaa ggagttggaa gtgaagtcta tgatgtgaaa |
| 1381 | cactttgcct cctgtgtact gtgtcataaa cagatgaata aactgaattt gtactttaaa |
| 1441 | aaaaaaaaaa aaaaaa |

SEQ ID NO 2

| | |
|---|---|
| 1 | MSTVHEILCK LSLEGDHSTP PSAYGSVKAY TNFDAERDAL NIETAIKTKG VDEVTIVNIL |
| 61 | TNRSNAQRQD IAFAYQRRTK KELASALKSA LSGHLETVIL GLLKTPAQYD ASELKASMKG |
| 121 | LGTDEDSLIE IICSRTNQEL QEINRVYKEM YKTDLEKDII SDTSGDFRKL MVALAKGRRA |
| 181 | EDGSVIDYEL IDQDARDLYD AGVKRKGTDV PKWISIMTER SVPHLQKVFD RYKSYSPYDM |
| 241 | LESIRKEVKG DLENAFLNLV QCIQNKPLYF ADRLYDSMKG KGTRDKVLIR IMVSRSEVDM |
| 301 | LKIRSEFKRK YGKSLYYYIQ QDTKGDYQKA LLYLCGGDD |

SEQ ID NO 3
AEDGSVIDYELIDQDAR
SEQ ID NO 4
AYTNFDAER
SEQ ID NO 5
AYTNFDAERDAL
SEQ ID NO 6
DAERDALNIET
SEQ ID NO 7
DALNIETAIK
SEQ ID NO 8
DALNIETAIKTK
SEQ ID NO 9
DIISDTSGDFR
SEQ ID NO 10
DLYDAGVK
SEQ ID NO 11
DLYDAGVKR
SEQ ID NO 12
EGDHSTPPSAYGSVK
SEQ ID NO 13
ELASALK
SEQ ID NO 14
ELIDQDARDL
SEQ ID NO 15
GDLENAFLNLVQCIQNKPLYFADR
SEQ ID NO 16
GLGTDEDSLIEIICSR
SEQ ID NO 17
GTDEDSLIEIICSR
SEQ ID NO 18
GVDEVTIVNI
SEQ ID NO 19
GVDEVTIVNIL
SEQ ID NO 20
GVDEVTIVNILTNR
SEQ ID NO 21
HSTPPSAYGSVKA
SEQ ID NO 22
HSTPPSAYGSVKAY
SEQ ID NO 23
LMVALAK
SEQ ID NO 24
LSLEGDHSTPPSAYGSVK
SEQ ID NO 25
NKPLYFADR
SEQ ID NO 26
QDIAFAYQR
SEQ ID NO 27
RAEDGSVIDYELIDQDAR
SEQ ID NO 28
SALSGHLETVIL
SEQ ID NO 29
SALSGHLETVILGLLK
SEQ ID NO 30
SEVDMLK
SEQ ID NO 31
SLYYYIQQDTK
SEQ ID NO 32
SLYYYIQQDTKGDYQK
SEQ ID NO 33
STPPSAYGSVKAY
SEQ ID NO 34
STPPSAYGSVKAYTNF
SEQ ID NO 35
STVHEILCK
SEQ ID NO 36
SYSPYDMLESIR
SEQ ID NO 37
TDLEKDIISDTSGDFR
SEQ ID NO 38
TNFDAERDALNIETAIKTK
SEQ ID NO 39
TNQELQEINR
SEQ ID NO 40
TPAQYDASELK
SEQ ID NO 41
WISIMTER
SEQ ID NO 42
YTNFDAER
SEQ ID NO 43
YIQQDTKGDYQKAL

1. A cosmetic method for preventing and/or treating the signs of skin dryness, comprising:
administering an effective amount of at least one polypeptide having an amino acid sequence encoded by a nucleic acid sequence wholly or partially represented by a sequence represented by SEQ ID No. 1, an analogue thereof or a fragment thereof, of at least one nucleic sequence encoding such a polypeptide or of at least one modulator of the activity, the stability or the expression of such a polypeptide.

2. A therapeutic method for preventing and/or treating the signs of skin dryness, comprising:
administering a therapeutic composition prepared with an effective amount of at least one polypeptide having an amino acid sequence encoded by a nucleic acid sequence wholly or partially represented by a sequence represented by SEQ ID No. 1, or an analogue thereof or a fragment thereof, of at least one nucleic sequence encoding such a polypeptide or of at least one modulator of the activity, the stability or the expression of such a polypeptide.

3. The method according to claim 1, in which said polypeptide has an amino acid sequence wholly or partially represented by a sequence represented by SEQ ID No. 2, or an analogue thereof or a fragment thereof.

4. The method according to claim 1, wherein said polypeptide has an amino acid sequence selected from the group consisting of SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42 and SEQ ID No. 43.

5. The method according to claim 1, wherein the at least one modulator is an inhibitor of the expression of said at least one polypeptide.

6. The method according to claim 1, wherein the at least one modulator is an agent for decreasing the stability of said at least one polypeptide.

7. The method according to claim 6, wherein said agent for decreasing the stability of said at least one polypeptide is a compound for stimulating proteolytic degradation, selected from the group consisting of proteases, ion chelators, sulphonic derivatives, urea derivatives, reducing agents, alpha- or beta-hydroxy acids, ascorbic acid and nicotinamide.

8. A method for characterizing, in vitro or ex vivo, a state of an epithelium, said state of the epithelium being a state of dryness, comprising:
determining at least one polypeptide having an amino acid sequence encoded by a nucleic acid sequence wholly or partially represented by a sequence represented by SEQ ID No. 1, an analogue thereof or a fragment thereof, or at least one nucleic acid sequence encoding said polypeptide.

9. A process for characterizing a state of dryness of an epithelium, comprising:
a) determining, in a sample of said epithelium, the content of a polypeptide as defined in any one of claims 1 to 4 or of a nucleic acid sequence encoding said polypeptide, and
b) comparing said content determined in a) to a reference value.

10. The process according to claim 9, wherein the process is noninvasive.

11. A cosmetic process for characterizing the effectiveness of a cosmetic or therapeutic treatment aimed at compensating for the signs of skin dryness, comprising at least the qualitative or quantitative characterization of the expression and/or of the biological activity of a polypeptide having an amino acid sequence encoded by a nucleic acid sequence wholly or partially represented by a sequence represented by SEQ ID No. 1, an analogue thereof or a fragment thereof.

12. The method according to claim 2, wherein said polypeptide has an amino acid sequence wholly or partially represented by a sequence represented by SEQ ID No. 2, or an analogue thereof or a fragment thereof.

13. The method according to claim 2, wherein said polypeptide has an amino acid sequence selected from the group consisting of SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42 and SEQ ID No. 43.

14. The method according to claim 2, wherein the modulator is an inhibitor of the expression of said polypeptide.

15. The method according to claim 2, wherein the modulator is an agent for decreasing the stability of said polypeptide.

16. The method according to claim 2, wherein said agent is a compound for stimulating proteolytic degradation, selected from the group consisting of proteases, ion chelators, sulphonic derivatives, urea derivatives, reducing agents, alpha- or beta-hydroxy acids, ascorbic acid and nicotinamide.


SEQUENCE LISTING (43 sequences)

SEQ ID NO: 1 - DNA, 1456 bp, Homo sapiens
gggacgctctcagctctcggcgcacggcccaggttatcttgtagcatagcaacttcggatttcactctacccggagagtttcccgcttggttgaacacattggcctcaggaagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttaaaaaaaaaaaaaaaaaaa

SEQ ID NO: 2 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 3 - protein, 17 aa, Homo sapiens
AEDGSVIDYELIDQDAR

SEQ ID NO: 4 - protein, 9 aa, Homo sapiens
AYTNFDAER

SEQ ID NO: 5 - protein, 12 aa, Homo sapiens
AYTNFDAERDAL

SEQ ID NO: 6 - protein, 11 aa, Homo sapiens
DAERDALNIET

SEQ ID NO: 7 - protein, 10 aa, Homo sapiens
DALNIETAIK

SEQ ID NO: 8 - protein, 12 aa, Homo sapiens
DALNIETAIKTK

SEQ ID NO: 9 - protein, 11 aa, Homo sapiens
DIISDTSGDFR

SEQ ID NO: 10 - protein, 8 aa, Homo sapiens
DLYDAGVK

SEQ ID NO: 11 - protein, 9 aa, Homo sapiens
DLYDAGVKR

SEQ ID NO: 12 - protein, 15 aa, Homo sapiens
EGDHSTPPSAYGSVK

SEQ ID NO: 13 - protein, 7 aa, Homo sapiens
ELASALK

SEQ ID NO: 14 - protein, 10 aa, Homo sapiens
ELIDQDARDL

SEQ ID NO: 15 - protein, 24 aa, Homo sapiens
GDLENAFLNLVQCIQNKPLYFADR

SEQ ID NO: 16 - protein, 16 aa, Homo sapiens
GLGTDEDSLIEIICSR

SEQ ID NO: 17 - protein, 14 aa, Homo sapiens
GTDEDSLIEIICSR

SEQ ID NO: 18 - protein, 10 aa, Homo sapiens
GVDEVTIVNI

SEQ ID NO: 19 - protein, 11 aa, Homo sapiens
GVDEVTIVNIL

SEQ ID NO: 20 - protein, 14 aa, Homo sapiens
GVDEVTIVNILTNR

SEQ ID NO: 21 - protein, 13 aa, Homo sapiens
HSTPPSAYGSVKA

SEQ ID NO: 22 - protein, 14 aa, Homo sapiens
HSTPPSAYGSVKAY

SEQ ID NO: 23 - protein, 7 aa, Homo sapiens
LMVALAK

SEQ ID NO: 24 - protein, 18 aa, Homo sapiens
LSLEGDHSTPPSAYGSVK

SEQ ID NO: 25 - protein, 9 aa, Homo sapiens
NKPLYFADR

SEQ ID NO: 26 - protein, 9 aa, Homo sapiens
QDIAFAYQR

SEQ ID NO: 27 - protein, 18 aa, Homo sapiens
RAEDGSVIDYELIDQDAR

SEQ ID NO: 28 - protein, 12 aa, Homo sapiens
SALSGHLETVIL

SEQ ID NO: 29 - protein, 16 aa, Homo sapiens
SALSGHLETVILGLLK

SEQ ID NO: 30 - protein, 7 aa, Homo sapiens
SEVDMLK

SEQ ID NO: 31 - protein, 11 aa, Homo sapiens
SLYYYIQQDTK

SEQ ID NO: 32 - protein, 16 aa, Homo sapiens
SLYYYIQQDTKGDYQK

SEQ ID NO: 33 - protein, 13 aa, Homo sapiens
STPPSAYGSVKAY

SEQ ID NO: 34 - protein, 16 aa, Homo sapiens
STPPSAYGSVKAYTNF

SEQ ID NO: 35 - protein, 9 aa, Homo sapiens
STVHEILCK

SEQ ID NO: 36 - protein, 12 aa, Homo sapiens
SYSPYDMLESIR

SEQ ID NO: 37 - protein, 16 aa, Homo sapiens
TDLEKDIISDTSGDFR

SEQ ID NO: 38 - protein, 19 aa, Homo sapiens
TNFDAERDALNIETAIKTK

SEQ ID NO: 39 - protein, 10 aa, Homo sapiens
TNQELQEINR

SEQ ID NO: 40 - protein, 11 aa, Homo sapiens
TPAQYDASELK

SEQ ID NO: 41 - protein, 8 aa, Homo sapiens
WISIMTER

SEQ ID NO: 42 - protein, 8 aa, Homo sapiens
YTNFDAER

SEQ ID NO: 43 - protein, 14 aa, Homo sapiens
YIQQDTKGDYQKAL